(19) United States
(12) Patent Application Publication
(10) Pub. No.: US 2026/0022223 A1
(43) Pub. Date: Jan. 22, 2026

(54) SUPERCRITICAL CARBON DIOXIDE FOR SHAPE MEMORY FOAM SYNTHESIS AND PROCESSING

(71) Applicant: Boston Scientific Scimed Inc., Maple Grove, MN (US)

(72) Inventors: Scott Andrew McCullough, Plymouth, MN (US); Patrick Willoughby, Shoreview, MN (US); Sasha Viola Rios, Minneapolis, MN (US); Paul Vincent Grosso, Maple Grove, MN (US)

(21) Appl. No.: 19/273,865

(22) Filed: Jul. 18, 2025

Related U.S. Application Data
(60) Provisional application No. 63/673,636, filed on Jul. 19, 2024.

(51) Int. Cl. C08J 9/38; A61L 2/20; A61L 2/235; C08J 9/12; C08J 9/14

(52) U.S. Cl. CPC C08J 9/38; A61L 2/20; A61L 2/235; C08J 9/125; C08J 9/142; C08J 2203/10; C08J 2203/12; C08J 2205/05; C08J 2375/04

(57) ABSTRACT

Methods for synthesizing and processing shape memory foams. The methods include utilizing supercritical carbon dioxide as a solvent, blowing agent, cleaning agent, sterilant, and/or reticulating agent.

### CROSS-REFERENCE TO RELATED PATENT APPLICATIONS

This application claims the benefit of priority to U.S. Provisional Patent Application No. 63/673,636, filed Jul. 19, 2024, the entire content and disclosure of which is incorporated herein by reference.

### TECHNICAL FIELD

The present disclosure relates generally to shape memory foams. More specifically, the disclosure relates to methods for synthesizing and processing shape memory foams using supercritical carbon dioxide and foams formed thereby.

### BACKGROUND

Shape memory foams are known in the art which exhibit at induction by an appropriate stimulus a shape transition from a temporary shape to a permanent shape corresponding to a previous programming. In some cases, this shape memory effect is thermally stimulated, that is, upon heating of the polymer material above the defined switching temperature which takes place by entropic elasticity recovery. Shape memory polymers are usually polymer networks at the molecular level, where chemical (covalent) or physical (non-covalent) crosslinks define the permanent shape.

These shape memory foams are useful in diverse applications like shape adaptive sportswear (e.g., helmets, suits), housing (i.e., thermal sealing of doors and windows) and robotics (i.e., conformal grip design). Also, these foams are being investigated for use in automobile and aerospace industries for self-healing automobile bodies and morphing aircraft wings. In addition, shape memory foam based biomedical devices are being developed. For example, shape memory foam based biomedical devices for minimally invasive surgeries are being developed.

Supercritical carbon dioxide (CO₂) is a fluid state of carbon dioxide where it is held at or above its critical temperature and critical pressure. Carbon dioxide behaves as a gas in air at standard temperature and pressure, or as a solid when cooled and/or pressurized sufficiently. If the temperature and pressure are both increased from standard temperature and pressure to be at or above the critical point for carbon dioxide, it can adopt properties midway between a gas and a liquid. Carbon dioxide will behave as a supercritical fluid above its critical temperature (304.128 K, 30.9780° C., 87.7604° F.) and critical pressure (7.3773 MPa, 72.808 atm, 1,070.0 psi, 73.773 bar).

Supercritical CO₂ has a variety of commercial and industrial uses due to its unique properties, such as its ability to diffuse through solids like a gas and dissolve materials like a liquid. In addition, supercritical CO₂ has relatively low toxicity and environmental impact compared to other industrial solvents. It has been used in extraction processes and catalysis, food processing, oil recovery, and cleaning applications.

### SUMMARY

Example 1 is a method for processing a shape memory foam, the method including a cleaning process, the cleaning process including: (i) applying a cleaning solvent composition comprising supercritical carbon dioxide to the shape memory foam; (ii) dissolving impurities from the shape memory foam into the cleaning solvent composition; and (iii) extracting the cleaning solvent composition including the dissolved impurities from the shape memory foam.

Example 2 is the method of Example 1 and further comprises purging and vaporizing the cleaning solvent composition containing the supercritical carbon dioxide after it is extracted from the shape memory foam.

Example 3 is the method of any of Examples 1-2, wherein the impurities comprise a catalyst, a surfactant, or a combination thereof.

Example 4 is the method of any of Examples 1-3, further comprising a synthesizing process before the cleaning process, the synthesizing process including: (i) synthesizing a polymer composition with a synthesis solvent composition comprising supercritical carbon dioxide in a vessel at a first temperature and a first pressure.

Example 5 is the method of any of Examples 1-4, further comprising a synthesizing and foam blowing process before the cleaning process, the synthesizing and foam blowing process including: (i) synthesizing a polymer composition with a synthesis solvent composition comprising supercritical carbon dioxide in a first vessel at a first temperature and a first pressure; and (ii) changing the polymer composition from the first temperature and the first pressure to a second temperature and a second pressure to produce the shape memory foam containing a plurality of pores distributed throughout the foamed material, wherein the first temperature and the second temperature are different, and wherein the first pressure and the second pressure are different.

Example 6 is the method of Example 5, wherein the synthesis solvent composition further comprises a blowing agent selected from acetone, isopropanol, ethanol, water, and combinations thereof.

Example 7 is the method of Example 5 or Example 6, wherein the polymer composition comprises a polyurethane polymer.

Example 8 is the method of any of Examples 1-7 wherein the impurities comprise a catalyst, a surfactant, or a combination thereof and the catalyst, the surfactant or the combination thereof is recycled to the synthesizing and foam blowing process.

Example 9 is the method of any of Examples 5-7, wherein second temperature and the second pressure occur in a second vessel.

Example 10 is the method of any of Examples 1-8, further comprising a sterilizing process after the cleaning process, the sterilizing process including: (i) contacting the shape memory foam with a sterilant comprising supercritical carbon dioxide.

Example 11 is the method of Example 10, wherein the sterilant further comprises a chemical sterilization additive selected from the group consisting of acetic acid, peracetic acid, trifluoroacetic acid, acetic acid derivatives, hydrogen peroxide and mixtures thereof.

Example 12 is a method for reticulating a foam, the method including: (i) permeating a reticulating agent comprising supercritical carbon dioxide into a foam having pores defined by membranes; and (ii) expanding the reticulating agent inside the foam to break the membranes enclosing the pores of the foam.

Example 13 is the method of Example 12 wherein the expansion of the reticulating agent inside the foam yields an open-cell morphology.

Example 14 is the method of Example 12 or Example 13, wherein expanding the reticulating agent includes depressurization.

Example 15 is the method of any of Examples 12-14, further including a synthesizing and foam blowing process with the following steps: (i) synthesizing a polymer composition in a solvent composition comprising supercritical carbon dioxide at a first temperature and a first pressure; and (ii) externally changing the polymer composition from said first temperature and said first pressure to a different second temperature and a second pressure to produce the foam having the pores, wherein the first temperature and the second temperature are different and wherein the first pressure and the second pressure are different.

Example 16 is the method of any of Examples 12-15, wherein the polymer composition comprises a polyurethane polymer.

Example 17 is the method of any of Examples 12-16, wherein the foamed material is a shape memory foam.

Example 18 is the method of any of Examples 12-17, further comprising a sterilizing process with the following step: (i) contacting the foam with a sterilant comprising supercritical carbon dioxide.

Example 19 is the method of Example 18, wherein the sterilant further comprises a chemical sterilization additive selected from the group consisting of acetic acid, peracetic acid, trifluoroacetic acid, acetic acid derivatives, hydrogen peroxide and mixtures thereof.

Example 20 is a method for cleaning and reticulating a foam, the method including: (i) applying a cleaning solvent composition comprising supercritical carbon dioxide to a foam having pores defined by membranes; (ii) dissolving impurities from the foam into the cleaning solvent composition; (iii) extracting the cleaning solvent composition comprising the dissolved impurities from the foam; (iv) permeating a reticulating agent comprising supercritical carbon dioxide into the foam; and (v) expanding the reticulating agent inside the foam to break the membranes enclosing the pores of the foam.

While multiple examples are disclosed, still other examples in accordance with this disclosure will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 is a flow chart of a process flow in accordance with one embodiment of the present disclosure.

FIG. 2 is a schematic of a continuous flow system in accordance with one embodiment of the present disclosure.

While this disclosure is amenable to various modifications and alternative forms, specific embodiments have been shown by way of example in the drawings and are described in detail below. The intention, however, is not to limit the disclosure to the particular embodiments described. On the contrary, this disclosure is intended to cover all modifications, equivalents, and alternatives falling within the scope of the disclosure as defined by the appended claims.

### DETAILED DESCRIPTION

Disclosed herein are methods for synthesizing and processing shape memory materials with supercritical carbon dioxide. The disclosed methods that utilize supercritical carbon dioxide reduce or even eliminate the numerous additives and complex post-synthesis processing steps typically needed to achieve a shape memory material desired chemical composition and physical morphology. Beneficially, the temperature and pressure of supercritical carbon dioxide can be controlled to alter its properties. Supercritical carbon dioxide can also be recovered after use and recycled, making it a more environmentally friendly alternative to conventional solvents.

Various types of foam, including but not limited to shape memory foam, may be used in a variety of different medical devices. Medical devices may be made from one or more pieces of foam that are secured together to create a more complex geometry, for example. Foam may be added to other components. Foam may be added to one or more surfaces of another component, such as metal surfaces, polymeric surfaces, or even other foam surfaces. In some cases, foam such as a shape memory foam may be used as a connecting element between other components of a device. Foam may be used for positioning one device component relative to another device component. Foam may be used to allow compression between components and/or to increase stiffness between components.

Shape memory foam manufacturing can be divided into three primary components: (I) synthesis and foaming, (II) subsequent cleaning and reticulation, and (III) sterilization. As described herein, supercritical caron dioxide can be utilized in one, two or all three of these components.

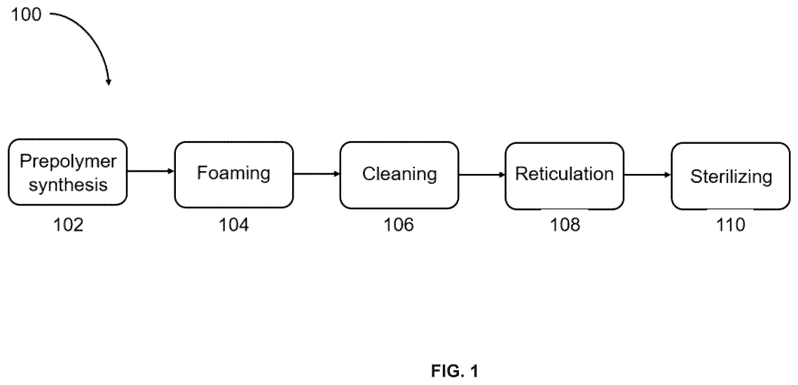

FIG. 1 is a flowchart of method 100 for making a shape memory foam which includes the following steps: prepolymer synthesis 102, foaming 104, cleaning 106, reticulation 108, and sterilizing 110.

### I. Synthesis and Foaming of Shape Memory Foams with Supercritical CO₂

Step 102 is prepolymer synthesis. Shape memory foams may be synthesized from a foamable solution that includes the monomers, catalysts and blowing agents that will react. The foamable solution may also include other materials such as surfactants.

At the molecular level, shape memory foams include a polymer network which has covalent cross-linking sites as well as switching segments which exhibit a transition temperature in an acceptable range for the respective application. These features allow shape memory foams to transform from a temporary shape to a permanent shape when triggered by an environmental stimulus such as heat, light, or vapor. Once the monomer and/or polymer is chosen and synthesized in prepolymer synthesis step 102, a foaming or blowing step occurs which incorporates air bubbles into the polymer matrix to form a foam material.

As an example, a shape memory foam may be composed of several components, including monomers, chemical blowing agents, physical blowing agents, surfactants, and catalysts. Chemical blowing agents undergo a chemical change to generate gas that helps to blow the foam. Chemical blowing agents may react with monomers in solution or chemically degrade to produce gas. Physical blowing agents undergo a physical change (liquid to vapor) to generate gas. In some cases, catalysts may facilitate polyurethane bond formation and some chemical reactions.

Examples of suitable monomers for making shape memory polymer foams include polyols and isocyanates. Examples of polyols that are suitable for making shape memory foam polymers include N,N,N′,N′-tetrakis(2-hydroxypropyl)ethylenediamine, triethanolamine, diethanolamine, dipropylene glycol, 5-amino-2,4,6-triiodoisophthalic acid, 3-methyl-1,5-pentanediol, gadopentetic acid, 2-butyl-2-ethyl-1,3-propanediol, and 1,2,6-hexanetriol. Examples of isocyanates that are suitable for making shape memory polymer foams include hexamethylene diisocyanate, 2,2,4- and 2,4,4-trimethylhexamethylene diisocyanate, isophorone diisocyanate, and 1,3-bis(isocyanatomethyl)cyclohexane.

Examples of suitable chemical blowing agents include, but are not limited to, water, sodium bicarbonate, and azodicarbonamide. Examples of suitable physical blowing agents include, but are not limited to, carbon dioxide, acetone, dimethoxymethane, methyl formate, ethanol, other high-vapor pressure solvents, or specialty commercial physical blowing agents. In some embodiments, a chemical and/or physical blowing agent can be used in combination with supercritical carbon dioxide. In some embodiments, the blowing agent is acetone, isopropanol, ethanol, water, and combinations thereof.

In some examples, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polyglyconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy) propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerization of various diketene acetals and diols; (d) polyanhydride homopolymers and copolymers such as poly(adipic anhydride), poly(suberic anhydride), poly(sebacic anhydride), poly(dodecanedioic anhydride), poly(maleic anhydride), poly[1,3-bis-(p-carboxyphenoxy) methane anhydride], and poly[alpha,omega-bis(p-carboxyphenoxy) alkane anhydride] such as poly[1,3-bis(p-carboxyphenoxy) propane anhydride] and poly[1,3-bis(p-carboxyphenoxy) hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly(D, L-lactide) (PDLLA), PVA, ethylene vinyl acetate copolymer, (EVA) polymer blend, polymer composites, crosslinked polymers, and supramolecular networks, among others. In some instances, shape memory polymers that may be used in creating the foamable solutions described herein may include polyurethane, for example.

In one embodiment, prepolymer synthesis includes synthesizing a polymer composition with a synthesis solvent composition comprising supercritical carbon dioxide in a vessel at a first temperature and a first pressure. In some embodiments, prepolymer synthesis step 102 may be conducted at a temperature from about −50° C. to about 250° C. or from about 0° C. to about 150° C. In some embodiments, prepolymer synthesis step 102 may be conducted at a pressure from about 0.0344 megapascal (MPa) (5 Pounds per Square Inch Absolute (psia)) to about 103.421 MPa (15000 psia) or from about 0.0690 MPa (10 psia) to about 41.369 MPa (6000 psia).

Synthetic procedures for synthesizing polyurethane shape memory foams are described in detail in Landsman et al., Design and Verification of a Shape Memory Polymer Peripheral Occlusion Device, Journal of the Mechanical Behavior of Biomedical Materials 63 (2016): 195-206, which is incorporated herein in its entirety. In general, the reaction first involves a prepolymer reaction between the isocyanate and polyol followed by reaction with any remaining or additional polyols, catalysts, and surfactants to produce the finished polyurethane.

In some embodiments, a synthesis solvent composition is used in the prepolymer synthesis which includes supercritical carbon dioxide (CO₂). In some embodiments, it may also include additional organic solvents such as toluene, xylene, ethyl acetate, dichloromethane, dimethylformamide, dimethylsulfoxide, tetrahydrofuran, methyl ethyl ketone, and combinations of the foregoing.

Prepolymer synthesis may be performed without the addition of a solvent. However, some monomers such as N, N,N′,N′-tetrakis(hydroxypropyl)ethylenediamine (HPED) may have a high viscosity, making them difficult to incorporate it into the prepolymer solution. Additionally, the difficultly of incorporating monomers such as HPED can create issues in the reaction due to localized differences in reagent concentrations. In comparison, supercritical carbon dioxide reduces the viscosity of the prepolymer mixture. This allows for better control of reagent concentrations (including reducing localized differences) and therefore modulating reaction rates of the prepolymer synthesis.

In one embodiment, in step 102, the reaction vessel may be charged with the polyols and isocyanate, then the synthesis solvent including supercritical carbon dioxide, which allows the polyols to dissolve and produce a homogenous prepolymer solution, is added. Advantageously, using supercritical carbon dioxide as a solvent allows for more precise control of the rate of reaction and reaction temperature of the prepolymer synthesis. The reduced reaction rate and improved mass/heat transfer aids in heat dissipation, thereby avoiding formation of undesirable byproducts in the synthesis.

Following synthesis step 102, the polymer mixture may be foamed in foaming step 104. In some embodiments, following the prepolymer synthesis, additional reagents such as the polyol, catalysts, surfactants, and water for foaming are added to the product of the prepolymer synthesis. The foaming step can also include changing the pressure and temperature of the reaction vessel. In some embodiments, prepolymer synthesis step 102 and foaming step 104 can be conducted in the same reaction vessel. In this situation, the temperature and pressure of the reaction vessel can be changed from the first temperature and pressure of the prepolymer synthesis step 102 to a second temperature and second pressure, which are different than the first pressure and the first temperature.

Examples of suitable catalysts include tin catalysts and amine catalysts. Examples of suitable tin catalysts include 2-ethylhexyl 4,4-dibutyl-10-ethyl-7-oxo-8-oxa-3,5-dithia-4-stannatetradecanoate, and Evonik DABCO® T131. Examples of suitable amine catalysts include 1,4-diazabicyclo[2,2,2]octane, 1,1,4,7,7-pentamethyldiethylenetriamine, 2,6,10-trimethyl-2,6,10-triazaundecane, bis[2-(N,N-dimethylamino)ethyl)]ether, Evonik DABCO® BL 11, and Evonik DABCO® BL 22.

In some cases, a foamable solution may include a surfactant such as a silicone surfactant such as that commercially available from Evonik under the name TEGOSTAB® B 8418. Other surfactants are also contemplated.

Changing the temperature and pressure of the reaction vessel allows the supercritical carbon dioxide in the solvent composition to vaporize, nucleating bubbles which expand within the foam. This process produces a shape memory foam containing a plurality of pores distributed throughout the foamed material.

The resulting shape memory foam may have a distribution of pore sizes in the range going from tens of nanometers to 1800 micrometers. The pores can be classified as small, medium, and large pores. In one example, small pores can have a pore size of up to 1000 micrometers, medium pores can have a pore size of greater than about 1000 micrometers to about 1500 micrometers, and large pores can have a pore size of greater than 1500 microns to about 1800 microns. In some embodiments, the shape memory foam can have an average pore diameter of about 1 millimeter.

In some embodiments, foaming step 104 may be conducted at a temperature from about −50° C. to about 250° C. or from about 0° C. to about 150° C. In some embodiments, foaming step 104 may be conducted at a pressure from about 0.0344 MPa (5 psia) to about 103.421 MPa (15,000 psia) or from about 0.0690 MPa (10 psia) to about 41.369 MPa (6000 psia). In some embodiments, the temperature of foaming step 104 and/or synthesis step 102 can be selected so that the temperature difference from synthesis step 102 to foaming step 104 is within a predetermined value. Additionally, or alternatively, in some embodiments, the pressure of foaming step 104 and/or synthesis step 102 can be selected so that the pressure difference from synthesis step 102 to foaming step 104 is within a predetermined value.

In some embodiments, prepolymer synthesis step 102 and foaming step 104 can be conducted in separate reaction vessels. In other embodiments, prepolymer synthesis step 102 and foaming step 104 can be conducted in the same reaction vessel.

II. Cleaning and Reticulation of Shape Memory Foams with Supercritical CO₂

The present disclosure provides methods for extracting impurities from shape memory foams using supercritical CO₂ as a solvent and for reticulating shape memory foams using supercritical CO₂ as a reticulating agent.

After synthesis (for example after the foaming step 104), shape memory foams may contain residual chemicals such as catalysts and surfactants which are left over from the polymerization. In cleaning step 106, the shape memory foam is cleaned using supercritical CO₂ as a solvent. For example, a cleaning solvent composition containing supercritical CO₂ can be applied to the shape memory foam. The application of the solvent composition may be done immediately or almost immediately after foaming step 104 when the foam is in a post-synthesis phase. In some embodiments, cleaning step 106 can occur in the same reaction vessel or a different reaction vessel as step 104. Alternatively, cleaning step 106 can be conducted after the shape memory foam has been cut to its desired geometry.

The supercritical CO₂ will dissolve one or more impurities in the shape memory foam. Then the solvent composition, which contains the dissolved impurities, is extracted from the shape memory foam. Example impurities include residual monomers, solvents, blowing agents, catalysts, and surfactants from prepolymer synthesis step 102 and foaming step 104. In some embodiments, supercritical CO₂ can be used in combination with a cosolvent for the extraction or dissolving of impurities in the shape memory foam. Example cosolvents include acetone, isopropanol, ethanol, water, and combinations thereof. Once the impurities have been extracted from the shape memory foam into the solvent composition, they may be recovered by purging the supercritical CO₂. For example, the supercritical carbon dioxide carrying extracted components can be transferred to a vessel at reduced pressure to allow extracted material to crash out of solution as the supercritical carbon dioxide vaporizes. In some embodiments, useful impurities such as catalysts may be recovered and recycled back into a process for synthesizing shape memory foams, for example at prepolymer synthesis step 102 and/or foaming step 104.

The cleaning methods described herein use supercritical CO₂ as a solvent, taking advantage of its high diffusivity through solids, and reducing the volume of conventional solvents needed to effectively wash foams. Advantageously, a shape memory foam cleaned using supercritical CO₂ will be dry without residual solvent, which helps maintain desired shape memory properties. Previous cleaning methods included alternative solvent extraction methods such as Soxhlet extraction, reflux extraction, high pressure solvent extraction.

Supercritical CO₂ may additionally or alternatively be used as a reticulating agent to create a more open-cell morphology in shape memory foams in reticulation step 108. In one embodiment, the method for reticulating shape memory foam includes permeating a reticulating agent including supercritical carbon dioxide into a foam; and expanding the reticulating agent inside the foam to break membranes enclosing the pores of the foam.

In some embodiments the reticulation process may be performed immediately or almost immediately after cleaning step 106. In some embodiments, cleaning step 106 and reticulation step 108 can be performed in the same reaction vessel. In other embodiments, cleaning step 106 and reticulation step 108 can be performed in different reactions vessels.

In its supercritical phase, carbon dioxide is able to readily permeate into the shape memory foam due to its low surface tension and high diffusivity. Once the reticulating agent including supercritical carbon dioxide has permeated the shape memory foam, the reaction vessel may be depressurized, allowing the carbon dioxide to expand in the closed-cells of the shape memory foam. This expansion breaks some or all of the thin membranes which define the pores of the shape memory foam to yield a shape memory foam having a more open open-cell morphology.

Rapid cooling as a result of the depressurization may also aid the reticulation process. For example, the rapid cooling of the shape memory foam can lower the material below or further below its glass transition temperature. This can result in a more brittle response of the shape memory foam which can increase the possibility that the membranes between the pores will break as the pores expand and contract. In some embodiments the expansion of the reticulation agent cools the foam. The extent of cooling of the shape memory foam can be controlled through heating the vessel while depressurizing. In some embodiments, the temperature drop of the shape memory foam could range from 20° C. to 100° C.

Once the thin membranes are broken, they may need be removed from inside the foam with an additional solvent cleaning step, which may be similar to cleaning step 106.

Controlled removal of the membranes of shape memory foams is useful for a variety of purposes including biomedical applications. Embodiments of the disclosed reticulation methods can be employed to control the quantity of membranes of shape memory foam that are pierced, leaving the base structure of the struts of the polymeric-based closed cell foam intact. The disclosed methods can be employed to pierce the membranes of the shape memory foam in one, two, or three dimensions.

III. Sterilizing Shape Memory Foams with Supercritical CO₂

The present disclosure provides methods for sterilizing shape memory foams using supercritical CO₂.

Referring to the flow in FIG. 1, after reticulation step 108, the shape memory foam is sterilized in step 110.

In one embodiment, sterilizing step 110 includes contacting the foamed material with a sterilant comprising supercritical carbon dioxide. In some embodiments, the sterilant may also include a chemical sterilization additive. Example chemical sterilization additives include acetic acid, peracetic acid, trifluoroacetic acid, acetic acid derivatives, hydrogen peroxide and mixtures thereof.

In some embodiments, sterilization may be enhanced by imparting turbulence or agitation to the sterilant either mechanically or by means of pressure cycling.

In some embodiments, the contact between the shape memory foam and sterilant is maintained for a sufficient time to achieve a 6-log reduction of microbes including at least one bacteria and/or virus.

Preferably, the sterilization process is conducted at a relatively low temperature, such as below the glass transition temperature, T_{g}, of the shape memory foam to prevent expansion of the shape memory foam in the programmed state.

The sterilization methods described herein are also well suited for the sterilization of thermally or hydrolytically sensitive, medically important materials, including biodegradable and other medical polymers, tissue for implantation or transplantation, medical equipment, drugs, and drug delivery systems. Advantageously, supercritical CO₂ can permeate into the shape memory foam and deactivate pathogens without exposing shape memory foams to high temperatures that can alter its chemical bonds. Proper sterilization of shape memory foams may be imperative in medical device applications for preventing infection and ensuring patient safety.

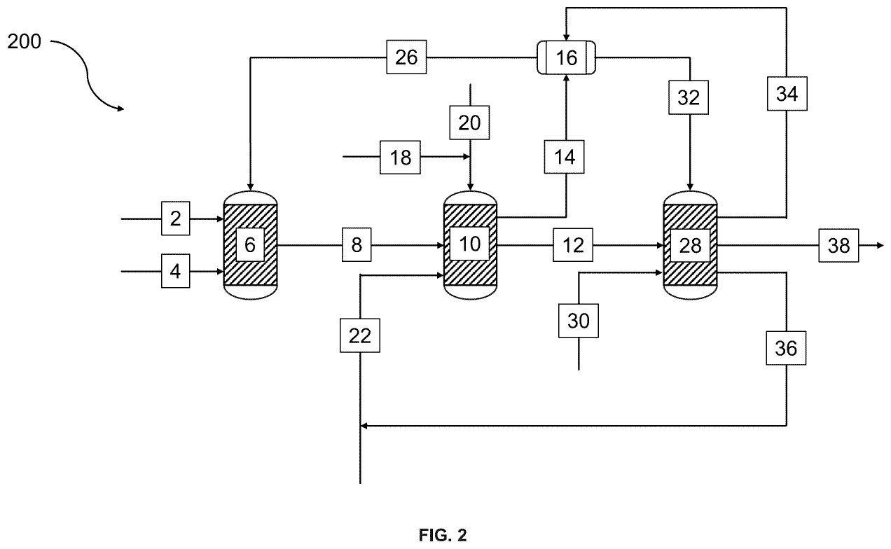

In some embodiments, one or more of the synthesis, cleaning and reticulation, and sterilization methods described herein may be combined into a continuous process. As used herein, the term “continuous process” refers to a method in which raw materials are continuously fed into the system, and products are continuously removed. Continuous processes can be advantageous because they can run non-stop, leading to more consistent product quality and higher efficiency. A schematic of one exemplary, continuous process is shown in FIG. 2. As shown therein, continuous process 200 includes reactor 6, reactor 10 and vessel 28. Stream 2 containing one or more isocyanates, stream 4 containing one or more polyols, and stream 26 containing a synthesis solvent composition comprising supercritical carbon dioxide are charged into reactor 6. Reactor 6 can be maintained at a first pressure and a first temperature as described herein.

After the prepolymer synthesis reaction is complete, the reaction mixture can be fed via stream 8 into reactor 10 where the foaming step occurs. In some embodiments, additional supercritical carbon dioxide can be fed into reactor 10. Reactor 10 is first charged with catalysts and surfactants from stream 22, water from stream 18, and polyols from stream 20 to complete a polyurethane reaction. Once the polyurethane reaction inside reactor 10 is completed, the raw polymer mixture, containing dissolved CO₂, is foamed by depressurizing the reactor. For example, reactor 10 can be maintained at a first pressure and temperature during the polyurethane reaction after which the pressure and temperature can be lowered, for example by releasing carbon dioxide gas from reactor 10. As the dissolved CO₂ nucleates and bubbles out of the solution, the gas forms bubbles within the polymer matrix. This expansion causes the polymer mixture to foam, creating a uniform cellular structure.

Carbon dioxide gas is released from reactor 10 via stream 14 and flows into CO₂ recycler 16. A shape memory foam is formed in reactor 10.

The shape memory foam from reactor 10 is transferred via stream 12 into vessel 28 where it can be washed and reticulated. Vessel 28 is also fed with supercritical CO₂ from stream 32 and solvents from stream 30. The shape memory foam from reactor 10 can include impurities or additives such as catalysts, surfactants, solvents and blowing agents. The shape memory foam from reactor 10 can also include unreacted monomers. Inside vessel 28, the leftover catalysts and surfactants from the synthesis process are extracted from the foam with supercritical CO₂ and removed in stream 36. Stream 36 may be recycled back into the process via stream 22. The foam can be reticulated by depressurizing vessel 28, allowing CO₂ gas to escape in stream 36 and flow back into CO₂ recycler 16. Finally, the clean final foam is removed from the process in stream 38.

Various modifications and additions can be made to the embodiments discussed without departing from the scope of this disclosure. For example, while the embodiments described above refer to particular features, the scope of this disclosure also includes embodiments having different combinations of features and embodiments that do not include all of the described features. Accordingly, the scope of this disclosure is intended to embrace all such alternatives, modifications, and variations as fall within the scope of the claims, together with all equivalents thereof.

1. A method for processing a shape memory foam, the method comprising a cleaning process, the cleaning process including:
(i) applying a cleaning solvent composition comprising supercritical carbon dioxide to the shape memory foam;
(ii) dissolving impurities from the shape memory foam into the cleaning solvent composition; and
(iii) extracting the cleaning solvent composition including the dissolved impurities from the shape memory foam.

2. The method of claim 1, further comprising purging and vaporizing the cleaning solvent composition containing the supercritical carbon dioxide after it is extracted from the shape memory foam.

3. The method of claim 1, wherein the impurities comprise a catalyst, a surfactant, or a combination thereof.

4. The method of claim 1, further comprising a synthesizing process before the cleaning process, the synthesizing process including:
(i) synthesizing a polymer composition with a synthesis solvent composition comprising supercritical carbon dioxide in a first vessel at a first temperature and a first pressure.

5. The method of claim 1, further comprising a synthesizing and foam blowing process before the cleaning process, the synthesizing and foam blowing process including:
(i) synthesizing a polymer composition with a synthesis solvent composition comprising supercritical carbon dioxide in a first vessel at a first temperature and a first pressure; and
(ii) changing the polymer composition from the first temperature and the first pressure to a second temperature and a second pressure to produce the shape memory foam containing a plurality of pores, wherein the first temperature and the second temperature are different, and wherein the first pressure and the second pressure are different.

6. The method of claim 5, wherein the synthesis solvent composition further comprises a blowing agent selected from acetone, isopropanol, ethanol, water, and combinations thereof.

7. The method of claim 5, wherein the polymer composition comprises a polyurethane polymer.

8. The method of claim 5, wherein the impurities comprise a catalyst, a surfactant, or a combination thereof and the catalyst, the surfactant or the combination thereof is recycled to the synthesizing and foam blowing process.

9. The method of claim 5, wherein the second temperature and the second pressure occur in a second vessel.

10. The method of claim 5 and further comprising a sterilizing process after the cleaning process, the sterilizing process including:
(i) contacting the shape memory foam with a sterilant comprising supercritical carbon dioxide.

11. The method of claim 10, wherein the sterilant further comprises a chemical sterilization additive selected from the group consisting of acetic acid, peracetic acid, trifluoroacetic acid, acetic acid derivatives, hydrogen peroxide and mixtures thereof.

12. A method for reticulating a foam, the method comprising:
(i) permeating a reticulating agent comprising supercritical carbon dioxide into a foam having pores defined by membranes; and
(ii) expanding the reticulating agent inside the foam to break the membranes enclosing the pores of the foam.

13. The method of claim 12, wherein expanding the reticulating agent inside the foam yields an open-cell morphology.

14. The method of claim 12, wherein expanding the reticulating agent includes depressurization.

15. The method of claim 12, further comprising a synthesizing and foam blowing process with the following steps:
(i) synthesizing a polymer composition in a solvent composition comprising supercritical carbon dioxide at a first temperature and a first pressure in a first vessel; and
(ii) externally changing the polymer composition from the first temperature and the first pressure to a second temperature and a second pressure to produce the foam having the pores, wherein the first temperature and the second temperature are different, and wherein the first pressure and the second pressure are different.

16. The method of claim 15, wherein the polymer composition comprises a polyurethane polymer.

17. The method of claim 12, wherein the foam is a shape memory foam.

18. The method of claim 12, further comprising a sterilizing process with the following step:
(i) contacting the foam with a sterilant comprising supercritical carbon dioxide.

19. The method of claim 18, wherein the sterilant further comprises a chemical sterilization additive selected from the group consisting of acetic acid, peracetic acid, trifluoroacetic acid, acetic acid derivatives, hydrogen peroxide and mixtures thereof.

20. A method for cleaning and reticulating a foam, the method comprising:
(i) applying a cleaning solvent composition comprising supercritical carbon dioxide to a foam having pores defined by membranes;
(ii) dissolving impurities from the foam into the cleaning solvent composition;
(iii) extracting the cleaning solvent composition comprising the dissolved impurities from the foam;
(iv) permeating a reticulating agent comprising supercritical carbon dioxide into the foam; and
(v) expanding the reticulating agent inside the foam to break the membranes enclosing the pores of the foam.
